SEQUENCE LISTING (5 sequences)

SEQ ID NO: 1 - protein, 1273 aa, Severe acute respiratory syndrome coronavirus 2
  MISC_FEATURE (342)..(342): Xaa = any amino acid
  MISC_FEATURE (354)..(354): Xaa = any amino acid
  MISC_FEATURE (367)..(367): Xaa = any amino acid
  MISC_FEATURE (435)..(435): Xaa = any amino acid
  MISC_FEATURE (458)..(458): Xaa = any amino acid
  MISC_FEATURE (483)..(483): Xaa = any amino acid
  MISC_FEATURE (683)..(683): Xaa = any amino acid
  MISC_FEATURE (685)..(685): Xaa = any amino acid
  MISC_FEATURE (986)..(987): Xaa = any amino acid
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVXNATRFASVYAWXRKRISNCVADYSXLYNSASFSTFKCYGVSPTKLNDLCFTNVYADSFVIRGDEVRQIAPGQTGKIADYNYKLPDDFTGCVIXWNSNNLDSKVGGNYNYLYRLFRXSNLKPFERDISTEIYQAGSTPCNGXEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRXAXSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDXXEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, Severe acute respiratory syndrome coronavirus 2; identical to SEQ ID NO: 1
  MISC_FEATURE (342)..(342): Xaa = any amino acid
  MISC_FEATURE (354)..(354): Xaa = any amino acid or Asx
  MISC_FEATURE (367)..(367): Xaa = any amino acid
  MISC_FEATURE (435)..(435): Xaa = any amino acid
  MISC_FEATURE (458)..(458): Xaa = any amino acid
  MISC_FEATURE (483)..(483): Xaa = any amino acid
  MISC_FEATURE (683)..(683): Xaa = any amino acid
  MISC_FEATURE (685)..(685): Xaa = any amino acid
  MISC_FEATURE (986)..(987): Xaa = any amino acid

SEQ ID NO: 3 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 805 aa, Homo sapiens [sequence omitted]

SEQ ID NO: 5 - protein, 958 aa, Artificial Sequence
  ACE2-Fc fusion protein
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIGGGGGGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0326240 A1
(43) Pub. Date: Oct. 13, 2022

(54) IMMUNOASSAY FOR SARS-COV-2 AND MATERIALS THEREFOR

(71) Applicant: BIO-RAD LABORATORIES, INC., HERCULES, CA (US)

(72) Inventors: RAVI KAUL, DAVIS, CA (US); SHUXIA ZHOU, BENICIA, CA (US); ROGER P. WALKER, BENICIA, CA (US)

(21) Appl. No.: 17/715,988

(22) Filed: Apr. 8, 2022

Related U.S. Application Data
(60) Provisional application No. 63/172,762, filed on Apr. 9, 2021.

(51) Int. Cl. G01N 33/569; G01N 33/573; G01N 33/62; G01N 33/533; G01N 1/40

(52) U.S. Cl. CPC G01N 33/56983; G01N 33/573; G01N 33/62; G01N 33/533; G01N 1/4044; G01N 2333/165; G01N 2800/26

(57) ABSTRACT

The present disclosure relates to the development of novel immunoassays for the detection of SARS-CoV-2 or secreted spike protein (or fragments thereof) in saliva, nasal mucosal sample, throat samples, or nasopharyngeal samples.

### CROSS-REFERENCE TO RELATED APPLICATION

This application claims the benefit of U.S. Provisional Application No. 63/172,762, filed Apr. 9, 2021, which is hereby incorporated by reference in its entirety.

### REFERENCE TO SEQUENCE LISTING

The Sequence Listing for this application is labeled “Seq-List.txt” which was created on Apr. 6, 2022 and is 49 KB. The entire content of the sequence listing is incorporated herein by reference in its entirety.

### BACKGROUND

SARS-CoV-2, a coronavirus, is the causative viral agent of the disease COVID-19 which is a highly infectious human respiratory infection that threatens global public health. As of February 2021 this virus was known to have infected at least 112.4 million people worldwide with at least 2,400,000 known deaths.

Coronavirus (CoV) is an enveloped virus that contains a single-stranded positive-sense RNA. SARS-CoV-2, formerly known as 2019-nCoV, is a newly emerging coronavirus that mainly affects the respiratory tract that can lead to Severe Acute Respiratory Syndrome (SARS). The underlying disease caused by this virus is named COVID-19. Coronaviruses have been responsible for several outbreaks in the world during the two last decades. In 2003 and 2014, coronaviruses caused outbreaks mainly in Asia (SARS-CoV) and in the Middle East (MERS-CoV), respectively. Before the emergence of the new SARS-CoV-2, six coronaviruses were known to affect humans (SARS-CoV, MERS-CoV and four other coronaviruses that cause mild upper and lower respiratory syndromes).

SARS-CoV-2 was first identified in December 2019, in Wuhan City, Hubei Province, China, after several patients developed severe pneumonia similar to that caused by SARS-CoV. The virus has since rapidly spread around the globe and in March 2020, WHO officially announced COVID-19 as a pandemic. Person to-person transmission of the virus resulted in quick spreading of COVID-19 and the high number of patients requiring intensive care resulted in the establishment of containment measures. Individuals infected with COVID-19 exhibit disease symptoms about 2 to 14 days after infection.

The virus has been detected in respiratory secretions, which are considered as the primary means of transmission. Once viral particles enter the respiratory tract, the virus attaches to pulmonary cells via the ACE-2 receptors (angiotensin-converting enzyme 2 (ACE2R)) and are then endocytosed. SARS-CoV-2 can also be transmitted via the fecal route.

Patients positive for SARS-CoV-2 and that are symptomatic are diagnosed with COVID-19. Symptoms can vary drastically and notably include fever, dry cough, anosmia, sputum production, headaches, dyspnea, fatigue, nausea, and diarrhea. While some cases can be asymptomatic, others can lead to acute respiratory distress syndrome (ARDS) that is associated with a “cytokine storm” and even death.

### BRIEF SUMMARY

The present disclosure relates to the development of novel immunoassays for the detection of SARS-CoV-2 infection. The immunoassays utilize a labeled ACE2 receptor (labeled with biotin or another label) that is admixed with a saliva, throat, nasal mucosal, or a nasopharyngeal sample from a subject (the first admixture). After admixing of the sample with labeled ACE2 receptor, a solid substrate to which RBD and/or spike protein (or variants thereof) is affixed (immobilized) is added to the first admixture mixture to form a second admixture. After a period of time, the solid substrate is separated from the other components of the second admixture and the binding of labeled ACE2 receptor to the solid substrate is determined. Depending on the amount of labeled ACE2 receptor bound to the solid substrate, the presence of SARS-CoV-2 or spike protein in the sample from the subject can be determined.

### BRIEF DESCRIPTION OF THE FIGURES

FIG. 1 illustrates one aspect of the disclosure in which ACE2R is mixed with a saliva, nasal mucosal, throat sample or a nasopharyngeal sample containing SARS-CoV2 and unbound ACE2R is detected using a solid substrate.

FIG. 2 and FIG. 3 illustrate signal inhibition by SARS-CoV-2 present in saliva samples obtained from patients confirmed to be infected by SARS-CoV-2 using RT-PCR.

### DETAILED DESCRIPTION

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

The present disclosure may refer to items, such as labels, solid supports, beads, analytes, etc. according to number or letter (e.g., Detectable label 1, bead (ii), etc.). Where this nomenclature is used, these numbers and letters are meant to distinguish the item from other items of the same type (e.g., bead (i) vs. bead (ii)), and are not meant to associate a specific property with the number or letter. Any methods, devices and materials similar or equivalent to those described herein can be used in the practice of this disclosure. The following definitions are provided to facilitate understanding of certain terms used frequently herein and are not meant to limit the scope of the present disclosure.

In the context of this application, the analyte to be measured is SARS-CoV-2.

The term “solid support” or “substrate” (and grammatical equivalents of these terms) are used to denote a solid inert surface or body to which an agent, such as an antibody or a peptide or protein can be immobilized or affixed. These terms (“solid support” or “substrate” (and grammatical equivalents of these terms)) may be used interchangeably. Non-limiting examples of a solid support or substrate include plastic, polystyrenes, nitrocellulose, membranes, chips, and particles. If solid supports other than particles are used, for instance, glass, polymeric or silica chips (such as microchips), plates, slides, etc., the peptides and/or proteins (target analytes) disclosed herein can be immobilized on the surface of the support at specific locations (e.g., in specific wells of a plate (e.g., microtiter plate) or at specific locations on a chip, microchip, plate or slide). Thus, it is possible to differentiate neutralizing antibodies specific for spike protein variants within a sample by the location at which specific binding between antibodies in a sample and the spike protein or fragments thereof occurs on the surface of the support.

The term “immobilized” as used herein denotes a molecular-based coupling that is not significantly de-coupled under the conditions imposed during the steps of the assays described herein. Such immobilization can be achieved through a covalent bond, a non-covalent bond, an ionic bond, an affinity interaction (e.g., avidin-biotin or polyhistidine-Ni⁺⁺), or any other chemical bond.

Immobilization of the spike protein, spike protein variants or fragments thereof (e.g., RBD) disclosed in this application can be performed by covalent or non-covalent immobilization on a substrate. For example, non-covalent immobilization can be non-specific (e.g., non-specific binding of a combination of one or more spike protein variants or fragments thereof to a polystyrene surface). Specific or semi-specific binding to a substrate can be achieved by the spike protein variants or fragments thereof having a moiety that enables covalent or non-covalent binding of the peptide and/or protein to the substrate that is coated with a ligand that binds to the moiety. For example, the moiety can be a biotin or biotinyl group or an analogue thereof bound to an amino acid group of the spike protein variants or fragment thereof, such as 6-aminohexanoic acid, and the ligand (biotin binding ligand) is avidin, streptavidin or an analogue thereof. Alternatively, the moiety can be a His-His-His-His-His-His peptide and the substrate can be derivatized with a Nitrilotriacetic Acid derivative (NTA) charged with Ni⁺⁺ ions.

Various substrates suitable for use in the disclosed methods include, and are not limited to, magnetic beads, polystyrene beads, latex beads, beads comprising co-polymers, such as styrene-divinyl benzene; hydroxylated styrene-divinyl benzene; polystyrene; carboxylated polystyrene; carbon black; non-activated, polystyrene or polyvinyl chloride activated glass; or epoxy-activated porous magnetic glass. In other embodiments, the substrate can be the floor or wall of a microtiter well; a filter surface or membrane (e.g., a nitrocellulose membrane or a PVDF (polyvinylidene fluoride) membrane, such as an Immobilon membrane); a hollow fiber; a beaded chromatographic medium (e.g., an agarose or polyacrylamide gel); a magnetic bead; a fibrous cellulose matrix; an HPLC matrix; an FPLC matrix; or any other suitable carrier, support or surface. In one embodiment of the disclosure, the disclosed SARS-CoV-2 spike protein variants or fragments thereof are immobilized onto polystyrene beads (microspheres), wherein each protein is immobilized onto a bead with a unique detectable physical parameter, and are analyzed by a platform capable of distinguishing the detectable physical parameter. Such beads can, optionally, also contain a magnetic core. Such assays may be referred to as “multiplex immunoassays” and are discussed in detail below.

Devices for performing specific binding assays, especially immunoassays, are known and can be readily adapted for use in the present methods. Solid phase assays, in general, are easier to perform than heterogeneous assay methods which require a separation step, such as precipitation, centrifugation, filtration, chromatography, or magnetism, because separation of reagents is faster and simpler. Solid-phase assay devices include microtiter plates, flow-through assay devices, chips, microchips, lateral flow substrates dipsticks and immunocapillary or immunochromatographic immunoassay devices.

The terms “receptacle,” “vessel,” “tube,” “well,” etc. refer to a container that can hold reagents or an assay. If the receptacle is in a kit and holds reagents, it will typically be closed or sealed. If the receptacle is being used for an assay, it will typically be open or accessible during steps of the assay.

The terms “patient sample” or “biological sample” encompasses a variety of sample types obtained from an organism, such as a human. In the context of the present disclosure, the biological sample is a saliva sample, throat sample, nasal mucosal sample, or a nasopharyngeal sample (e.g. a nasopharyngeal swab). The biological sample can be processed prior to assay, e.g., to remove cells or cellular debris. The term encompasses samples that have been manipulated after their procurement, such as by treatment with reagents, solubilization, sedimentation, or enrichment for certain components.

The terms “label,” “detectable label, “detectable moiety,” and like terms refer to a composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA), biotin, enzymes acting on a substrate (e.g., horseradish peroxidase), digoxigenin, ³²P and other isotopes, haptens, and proteins which can be made detectable, e.g., by incorporating a radiolabel into the peptide or used to detect antibodies specifically reactive with the peptide. The term includes combinations of single labeling agents, e.g., a combination of fluorophores that provides a unique detectable signature, e.g., at a particular wavelength or combination of wavelengths. Any method known in the art for conjugating label to a desired agent may be employed, e.g., using methods described in Hermanson, Bioconjugate Techniques 1996, Academic Press, Inc., San Diego.

The term “positive,” when referring to a result or signal, indicates the presence of an analyte or item that is being detected in a sample. The term “negative,” when referring to a result or signal, indicates the absence of an analyte or item that is being detected in a sample. With respect to the detection of SARS-CoV-2, spike protein, or fragments thereof in a biological sample according to this disclosure, a reduced signal, relative to a control in which no SARS-CoV-2 is present indicates the presence of SARS-CoV2, spike protein, or fragments thereof in the biological sample. Positive and negative are typically determined by comparison to at least one control, e.g., a threshold level that is required for a sample to be determined positive, or a negative control (e.g., a known blank). A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. For the assays used in the subject disclosure, control beads can be included, for example, a serum verification bead (SVB) and/or an internal standard bead (ISB) can be used. The lack of SVB signal confirms that serum and/or plasma sample has not been introduced into the reaction instead of the saliva, throat sample, nasal mucosal sample, or the nasopharyngeal sample and the ISB is provided in each receptacle to standardize detector performance. One of skill in the art will recognize that controls can be d/signed for assessment of any number of parameters, and will understand which controls are valuable in a given situation and be able to analyze data based on comparisons to control values. Controls are also valuable for determining the significance of data. For example, if values for a given parameter are variable in controls, variation in test samples will not be considered as significant.

A “calibration control” is similar to a positive control, in that it includes a known amount of a known analyte. In the case of a multiplex assay, the calibration control can be designed to include known amounts of multiple known analytes. The amount of analyte(s) in the calibration control can be set at a minimum cut-off amount, e.g., so that a higher amount will be considered “positive” for the analyte(s), while a lower amount will be considered “negative” for the analyte(s). In some cases, multilevel calibration controls can be used, so that a range of analyte amounts can be more accurately determined. For example, an assay can include calibration controls at known low and high amounts, or known minimal, intermediate, and maximal amounts.

“Subject,” “patient,” “individual” and grammatical equivalents thereof are used interchangeably and refer to, except where indicated, mammals, such as humans and non-human primates, as well as rabbits, felines, canines, rats, mice, squirrels, goats, pigs, deer, and other mammalian species. The term does not necessarily indicate that the subject has been diagnosed with a particular disease, but typically refers to an individual under medical or veterinary supervision. A patient can be an individual that is seeking treatment, monitoring, adjustment or modification of an existing therapeutic regimen, etc. In the context of this application, the “subject,” “patient,” or “individual” is suspected of having been exposed to or infected with SARS-CoV-2. The immunoassays disclosed herein are capable of detecting SARS-CoV-2, spike protein or fragments thereof in biological samples from these subjects.

As used herein, a “chaotropic agent” or “chaotrope” refers to a chemical compound that destabilizes the three-dimensional structure of proteins. In certain embodiments, a chaotropic agent may refer to an ionic chaotrope (e.g., a chaotropic ion or a chaotropic salt) or, alternatively, to a nonionic chaotrope. Non-limiting examples of chaotropic salts include: guanidinium salts, e.g., guanidinium chloride, guanidinium nitrate, guanidinium thiocyanate; thiocyanate salts, e.g., ammonium thiocyanate, potassium thiocyanate, sodium thiocyanate, lithium thiocyanate, calcium thiocyanate, guanidinium thiocyanate; perchlorate salts, e.g., ammonium perchlorate, sodium perchlorate, lithium perchlorate, magnesium perchlorate, calcium perchlorate; iodate salts, e.g., ammonium iodate, potassium iodate, sodium iodate, lithium iodate, magnesium iodate, calcium iodate; chlorate salts, e.g., sodium chlorate, lithium chlorate, magnesium chlorate, calcium chlorate; chloride salts, e.g., sodium chloride, potassium chloride, calcium, chloride, and ammonium chloride. Nonionic chaotropes include, without limitation, urea and thiourea. In non-limiting embodiments, the immunoassay disclosed herein can be conducted under high salt conditions (e.g., 1 M NaCl) or low salt conditions (e.g., 0.15 M NaCl). For the purposes of this disclosure as it relates to the presence of NaCl, the term “high salt” refers to conditions in which NaCl is present in an amount of at least 0.5 M to about 2.0 M, preferably about 0.75 M to about 1.25 M or about 1.0 M, and the term “low salt” refers to conditions in which NaCl is present in an concentration of 0.0 M to 0.5 M, preferably between about 0.1 M and about 0.2 M or about 0.15 M. In certain embodiments, low salt conditions are used for the assay.

The term “ACE2 receptor(s)” (ACE2R) includes truncated and/or modified ACE2 receptors that can have one, two, three, four, five, ten, 50, 100, 250, 500 or more amino acids removed, added, and/or substituted. A modified ACE2 receptor includes an ACE2 receptor fusion protein in which the ACE2 receptor, or a fragment or variant thereof is fused to an immunoglobulin Fc domain. For example, the Fc domain can be fused to the C-terminus of the ACE2 receptor, or a fragment or variant of the ACE2 receptor to form an ACE2R-Fc fusion protein. The fragment or variant of the ACE2 receptor can comprise a minimal domain from ACE2R that is sufficient to bind to the RBD, for example amino acids Ser19-Asp615 of ACE2R or amino acids 12-327 of the ACE2R. In various embodiments, human ACE2R (SEQ ID NO: 4) can be used. A “labeled ACE2 receptor” includes an ACE2 receptor that is detectably labeled (for example, with PE) or is a biotinylated, avidinated, or streptavidinated ACE2 receptor (including truncated and/or modified ACE2 receptors or ACE2 fusion proteins). In various embodiments, ACE2R-Fc fusion proteins can be biotinylated, avidinated, or streptavidinated on the Fc portion of the fusion protein.

The terms “structural protein” “peptide”, “antigen”, “analyte”, and “fragment” (and grammatical equivalents thereof) can be used interchangeably and refer to the disclosed SARS-CoV-2 spike proteins or fragments thereof that are disclosed herein. The term “SARS-CoV-2 spike proteins or fragments thereof” include the spike protein variants and fragments thereof as well as the unmutated SARS-CoV-2 spike protein (SEQ ID NO: 3) or fragments thereof that are disclosed herein (SEQ ID NO: 3). As discussed herein, fragments of the disclosed SARS-CoV-2 spike protein, SARS-CoV-2 spike protein variants are between 5 and (n−1) consecutive amino acids of a given SARS-CoV-2 spike protein (SEQ ID NO: 1 or SEQ ID NO: 2 or SEQ ID NO: 3). In each instance, a fragment of the spike protein variant will include, within its span of consecutive amino acids, the amino acid mutation associated with the mutant spike protein (which are identified in Table 1, Table 2 and Table 3). The numbering of the amino acid mutation is in relation to the amino acid numbering in SEQ ID NOs: 1 or 2 for Tables 1-3. Also within the scope of this disclosure are the spike protein sequences and fragments thereof, including RBD sequences, for various SARS-CoV-2 variants, such as B.1.1.7, B.1.351, P.1, and other known variants of SARS-CoV-2.

Also included as a spike protein variant are the spike proteins, RBD sequences, S1 and/or S2 fragments of the SARS-CoV-2 variants having the designations B.1.1.7 (UK), B.1.351 (South Africa), and P.1 (Brazil and Japan). Sequences for these spike protein variants are available in public databases. Table 3 also provides the amino acid mutations associated with these variants, relative to the wild type-sequence (SEQ ID NO: 3).

TABLE 1
Spike protein variants

| | Amino | | |
|---|---|---|---|
| | acid | aa | AA |
| Protein | mutation | range | substitution |
| S1 | 367 | 319-541 | Any amino acid, preferably F, L, |
| | | | A, V, I, M, Y or W |
| S1 | 342 | 319-541 | Any amino acid, preferably L, A, |
| | | | V, I, M, F, Y or W |
| S1 | 435 | 319-541 | Any amino acid, preferably S, T, |
| | | | N or Q |
| S1 | 458 | 319-541 | Any amino acid, preferably R, H |
| | | | or K |
| S1 | 483 | 319-541 | Any amino acid, preferably A, L, |
| | | | V, I, M, F, Y or W |
| S1 | 354 | 319-541 | Any amino acid, preferably D or |
| | | | E |
| S1/S2 | 683, 685, 986, and/or 987 | 1-1213 | Any amino acid, preferably L, A, |
| | | | V, I, M, F, Y, G, P or W |

TABLE 2
Preferred Spike protein variants

| | Mutant | AA | ACE2 |
|---|---|---|---|
| Protein | Type | Range | Binding |
| S1 | V367F | aa 319-541 | + |
| S1 | F342L | aa 319-541 | + |
| S1 | A435S | aa 319-541 | + |
| S1 | K458R | aa 319-541 | + |
| S1 | V483A | aa 319-541 | + |
| S1 | N354D | aa 319-541 | + |
| S1/S2 | R683A, R685A, K986P, V987P | aa 1-1213 | + |

TABLE 3
Spike protein variants for SARS-CoV2 variants B.1.351, B.1.1.7, and P.1

| | | | |
|---|---|---|---|
| S1; | 417, 484, and/or 501 | 319-541 | Any amino acid, preferably N, K, |
| B.1.351 | | | or Y, more preferably K417N, |
| | | | E484K and/or N501Y, even more |
| | | | preferably K417N, E484K and |
| | | | N501Y |
| S1/S2; | 417, 484, 501, 614 and/or | 1-1213 | Any amino acid, preferably N, K, |
| B.1.351 | 701 | | G, V or Y, more preferably |
| | | | K417N, E484K, N501Y, D614G, |
| | | | and/or A701V, even more |
| | | | preferably K417N, E484K, |
| | | | N501Y, D614G, and A701V |
| S1/S2; | Δ69H, Δ70V, Δ144Υ, 501, | 1-1213 | Any amino acid, preferably Y, D, |
| B.1.1.7 | 570, 614, 681, 761, 982, | | G, H, I, A, or H, more preferably |
| | and/or 1118 | | N501Y, A570D, D614G, P681H, |
| | | | T761I, S982A, and/or D1 118H, |
| | | | even more preferably N501Y, |
| | | | A570D, D614G, P681H, T761I, |
| | | | S982A, and D1118H |
| S1; P1 | 417, 484, and/or 501 | 319-541 | Any amino acid, preferably T, K, |
| | | | Y, more preferably K417T, |
| | | | E484K, and/or N501Y, even more |
| | | | preferably K417T, E484K, and |
| | | | N501Y |
| S1/S2; P1 | 18, 20, 26, 138, 190, 417, | 1-1213 | Any amino acid, preferably F, N, |
| | 484, 501, 614, 655, and/or | | S, Y, T, K, G, or I, more |
| | 1027 | | preferably L18F, T20N, P26S, |
| | | | D138Y, R190S, K417T, E484K, |
| | | | N501Y, D614, H655Y, and/or |
| | | | T1027I, even more preferably |
| | | | L18F, T20N, P26S, D138Y, |
| | | | R190S, K417T, E484K, N501Y, |
| | | | D614, H655Y, and/or T1027I |

With respect to the amino acid mutations at positions 683, 685, 986, and 987, any combination of 2, 3 or 4 amino acid mutations can exist in the spike protein variant or a fragment thereof. In certain embodiments, three or all four amino acid mutations exist within the spike protein variant or fragment thereof. Thus, fragments of the spike protein variants that can be immobilized on a solid support are fragments of S1 and can be between 5 and 685 consecutive amino acids in length, provided that the fragment includes one or more of the amino acid substitutions identified in Table 1 or 2 or 3 (for example, amino acids 13-685 or amino acids 319-541 of SEQ ID NO: 1 or 2 or 3). Preferred embodiments provide for spike protein variants, or fragments thereof, that contain a single amino acid substitution as identified in Tables 1 and 2 or the spike variants identified in Table 3. The length or a fragment can include or exclude signal peptides that are processed (for example amino acids 1-12 of SEQ ID NO: 1 or SEQ ID NO: 2). In some embodiments, the amino acid at position 354 of SEQ ID NO: 2 is Asx (aspartic acid or asparagine). Spike protein variants of SEQ ID NO: 2, thus, can contain either aspartic acid or asparagine at position 354 and one or more other mutations identified in Tables 1 and 2 (i.e., at positions 342, 367, 435, 458, 483, 683, 685, 986, and/or 987). Thus, in the case of the spike protein variants, the length can include or exclude the signal peptide of the protein.

One embodiment provides an immunoassay in a biological sample selected from saliva, a throat sample or a nasopharyngeal sample is contacted with labeled ACE2 receptors. This contacting step can be performed in the absence of a salt or chaotrope, under high salt or low salt conditions, or in the presence of a chaotrope under high salt or low salt conditions or in the presence of another chaotrope. After a predetermined period of time, a substrate to which a SARS-CoV-2 spike protein or fragment thereof has been immobilized is added to the mixture comprising the biological sample (saliva, a throat sample or a nasopharyngeal sample) and labeled ACE2R under conditions effective to permit the binding of labeled ACE2R to the solid substrate to which a SARS-CoV-2 spike protein or fragment thereof has been immobilized. Similar to the first contacting step, this step can also be conducted in the absence of a salt or chaotrope, under high salt or low salt conditions, or in the presence of a chaotrope. The solid substrate is then separated from the biological sample and the solid substrate can, optionally, be washed. ACE2R bound to the solid substrate can then be detected. As discussed above, the presence of SARS-CoV-2, a spike protein capable of binding ACE2R or a fragment of the spike protein capable of binding to ACE2R in a sample is determined by a decreased signal (or the absence of a signal) from ACE2R bound to the spike proteins or fragments thereof that are immobilized on a solid substrate.

In certain embodiments, labeled ACE2 receptor, including truncated and/or modified ACE2 receptors and/or ACE2R-fusion proteins (e.g., ACE2R-Fc fusion proteins) that are labeled are used to detect substrates to which SARS-CoV-2 spike protein or fragments thereof are immobilized. The truncated and/or modified ACE2 receptor can have one, two, three, four, five, ten, 50, 100, 250, 500 or more amino acids removed, added, and/or substituted. Alternatively, the ACE2 receptor can be fused to an immunoglobulin Fc domain. For example, the Fc domain can be fused to the C-terminus of ACE2R, or a fragment or variant of the ACE2 receptor protein. The fragment or variant of the ACE2 receptor comprises a minimal domain from ACE2R that is sufficient to bind to the RBD. Additionally, the ACE2 receptor, truncated or modified ACE2 receptor or an ACE2R fusion protein (e.g., ACE2R-Fc) can be biotinylated or avidinated. In preferred embodiments, the Fc domain of an ACE2R-Fc fusion protein can be biotinylated or avidinated. In such embodiments, it is possible to identify the presence of SARS-CoV-2 in a biological sample by the lack of labeled ACE2 receptor binding (or reduced ACE2R binding) to a substrate to which a SARS-CoV-2 spike protein or fragment thereof has been immobilized relative to a control sample that contains no SARS-CoV-2.

The SARS-CoV-2 spike protein or fragment thereof disclosed herein can be immobilized on a solid support via covalent or non-covalent bonding. In embodiments where a SARS-CoV-2 spike protein or fragment thereof is covalently immobilized on the substrate, carboxylated substrates, such as particles, plastics, polystyrenes or beads, are activated and esterified before adding the SARS-CoV-2 spike protein variant or fragment thereof or cytokine. Carboxyl activation is achieved using a water soluble carbodiimide, such as 1-cyclohexyl-3-(2-morpholinoethyl) carbodiimide (CMC). Esterification is achieved using NHS, NHSS or HOBt or other suitable reagents. After carboxyl activation and esterification, the SARS-CoV-2 spike protein or fragment thereof is added to the actived surface in buffers with pH between 6-10 (an example of which is sodium acetate buffer pH 5.1, phosphate buffer pH 7.0 with or without detergent (e.g., CHAPS)). After the coupling, the substrate (e.g., beads) is blocked in buffers containing protein blockers, such as BSA, mouse IgG, bovine gamma globulin (BGG) or animal serum (goat, horse, murine). The protein blocker(s) can be present in an amount ranging from 0.1-10 weight/volume percent. The blocked antigen coupled substrates can then be washed with an appropriate buffer and used in a desired immunoassay format.

As discussed above, the disclosed disclosure is directed to an immunoassay which includes taking a biological sample selected from a saliva, throat, or a nasopharyngeal swab likely to contain SARS-CoV-2 or a spike protein or fragment thereof; contacting (reacting) the biological sample with labeled ACE2R under conditions effective for the formation of a specific interaction between SARS-CoV-2 (or spike protein or fragment thereof) and the labeled ACE2R for form a first mixture; adding a substrate comprising immobilized SARS-CoV-2 spike protein or fragment thereof to the first mixture to form a second mixture; separating the solid substrate from the components of the second mixture; optionally washing the solid substrate to remove non-specifically associated protein from the solid substrate, and assaying the solid substrate for the presence of ACE2R bound to SARS-CoV-2 spike protein or fragments thereof bound to the solid substrate. As discussed above, any of the contacting/mixing steps can be performed under high salt or low salt conditions. The biological sample can be obtained from a SARS-CoV2 infected individual or suspected of being infected by SARS-CoV-2.

The presently described assays involve the use of a solid support, typically particles or beads. For detection by flow cytometry, particles or beads that emit high levels of autofluorescence should be avoided since this will increase background signal and potentially render them unsuitable. Particles or beads created by standard emulsion polymerization from a variety of starting monomers generally exhibit low autofluorescence, while those that have been modified to increase porosity (“macroporous” particles) exhibit high autofluorescence. Autofluorescence in such particles or beads further increases with increasing size and increasing percentage of divinylbenzene monomer. Within these limitations, the size range of the particles or beads can vary and particular size ranges are not critical. In most cases, the aggregated size range of the particles or beads lies within the range of from about 0.3 micrometer to about 100 micrometers in particle or bead diameter, e.g., within the range of from about 0.5 micrometer to about 40 micrometers.

Magnetic particles or beads are commonly used in the art, and can make separation and wash steps more convenient for the presently described assays. “Magnetic particles,” “magnetically responsive material,” “magnetic beads,” and like terms denote a material that responds to a magnetic field. Magnetically responsive materials include paramagnetic materials (e.g., iron, nickel, and cobalt, as well as metal oxides, such as Fe₃O₄, BaFe₁₂O₁₉, CoO, NiO, Mn₂O₃, Cr₂O₃, and CoMnP), ferromagnetic materials, ferrimagnetic materials, and metamagnetic materials. Rather than constituting the entire particle or bead, the magnetically responsive material typically constitutes one component of the microparticle or bead, while the remainder consists of a polymeric material which can be chemically derivatized to permit attachment of an assay reagent (e.g., antigen/analyte or antibody). Methods of, and instrumentation for, applying and removing a magnetic field as part of an assay are known to those skilled in the art and reported in the literature. Examples of literature reports are Forrest et al., U.S. Pat. No. 4,141,687; Ithakissios, U.S. Pat. No. 4,115,534; Vlieger et al., Analytical Biochemistry 205:1-7 (1992); Dudley, Journal of Clinical Immunoassay 14:77-82 (1991); and Smart, Journal of Clinical Immunoassay 15:246-251 (1992).

The polymeric matrix that forms the microparticle or bead can be any material that is compatible with the presently described multiplex assay. The matrix should be inert to the components of the biological sample (a saliva, throat, or a nasopharyngeal swab) and to the assay reagents, have minimal autofluorescence, be solid and insoluble in the sample and in any other reagents or washes used in the assay, and capable of affixing (immobilizing) an assay reagent to the microparticle. Examples of suitable polymers are polyesters, polyethers, polyolefins, polyalkylene oxides, polyamides, polyurethanes, polysaccharides, celluloses, and polyisoprenes. Crosslinking is useful in many polymers for imparting structural integrity and rigidity to the microparticle.

Functional groups for attachment of the assay reagent (e.g., antigen/analyte or antibody) can be incorporated into the polymer structure by conventional means. Examples of suitable functional groups are amine groups, ammonium groups, hydroxyl groups, carboxylic acid groups, and isocyanate groups. The assay reagent is typically covalently bound to the solid phase surface, either directly or indirectly, e.g., with a linking group. Linking groups can be used as a means of increasing the density of reactive groups on the solid phase surface and decreasing steric hindrance to increase the range and sensitivity of the assay, or as a means of adding specific types of reactive groups to the solid phase surface to broaden the range of types of assay reagents that can be affixed (immobilized) to the solid phase. Examples of suitable useful linking groups are polylysine, polyglycine, polyaspartic acid, polyglutamic acid and polyarginine.

Particles or beads of different types in a multiplex assay can be distinguished from one another, e.g., by size, weight, light scatter or absorbance, reflectance, shape, or label, e.g., fluorescent (dye) label. Where particle or bead size is used as a differentiation factor (distinguishing characteristic), the widths of the size subranges and the spacing between mean diameters of adjacent subranges are selected to permit differentiation of different types of particles or beads by flow cytometry, as will be apparent to those skilled in the use of and instrumentation for flow cytometry. Typically, a subrange for a given mean diameter is about ±5% CV or less of the mean diameter, where CV is the coefficient of variation and is defined as the standard deviation of the particle or bead diameter divided by the mean particle diameter times 100 percent. The mean diameters of subranges for different types of particles are generally spaced apart by at least about 6% of the mean diameter of one of the subranges, e.g., at least about 8% or 10% of the mean diameter of one of the subranges.

Light scatter can also be used to distinguish different types of particles or beads. Side angle light scatter varies with particle or beads size, granularity, absorbance and surface roughness, while forward angle light scatter is mainly affected by size and refractive index. Varying any of these qualities can result in light scatter differences that can serve as a means of distinguishing the various groups of particles or beads.

Still another example of a differentiation parameter is absorbance. When light is applied to particles or beads, the absorbance of the light by the particles or beads is indicated mostly by a change in the strength of the laterally (side-angle) scattered light while the strength of the forward-scattered light is relatively unaffected. Consequently, the difference in absorbance between various colored dyes associated with the particles or beads is determined by observing differences in the strength of the laterally scattered light.

Other physical parameters that can be used as differentiation parameters to distinguish the particles or beads of one group from those of another include excitable fluorescent dyes or colored dyes that impart different emission spectra and/or scattering characteristics to the particles or beads. Alternatively, different concentrations of one or more fluorescent dyes can be used for distinguishing or differentiating particles or beads.

When the distinguishable characteristic is a fluorescent dye or color, it can be coated on the surface of the particle or bead, embedded in the particle or bead, or bound to the molecules of the particle or bead material. Thus, fluorescent particles or beads can be manufactured by combining the polymer material with the fluorescent dye, or by impregnating the particle or bead with the dye. Particles or beads with dyes already incorporated and thereby suitable for use in the present disclosure are commercially available, from suppliers, such as Spherotech, Inc. (Libertyville, Ill., USA) and Molecular Probes, Inc. (Eugene, Oreg., USA).

Labels can be any substance or component that directly or indirectly emits or generates a detectable signal. In some embodiments, the labels are fluorophores, many of which are reported in the literature and thus known to those skilled in the art, and many of which are readily commercially available. Literature sources for fluorophores include Cardullo et al., Proc. Natl. Acad. Sci. USA 85: 8790-8794 (1988); Dexter, J. of Chemical Physics 21: 836-850 (1953); Hochstrasser et al., Biophysical Chemistry 45: 133-141 (1992); Selvin, Methods in Enzymology 246: 300-334 (1995); Steinberg, Ann. Rev. Biochem., 40: 83-114 (1971); Stryer, Ann. Rev. Biochem. 47: 819-846 (1978); Wang et al., Tetrahedron Letters 31: 6493-6496 (1990); and Wang et al., Anal. Chem. 67: 1197-1203 (1995). The following are non-limiting examples of fluorophores that can be used as labels:

4-acetamido-4′-isothiocyanatostilbene-2,2′disulfonic acid; acridine; acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl)maleimide; anthranilamide; BODIPY; Brilliant Yellow; coumarin; 7-amino-4-methylcoumarin (AMC, Coumarin 120); 7-amino-4-trifluoromethylcoumarin (Coumaran 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2, T-disulfonic acid; 5-[dimethylamino]naphthalene-1-sulfonyl chloride (DNS, dansylchloride); 4-(4′-dimethylaminophenylazo)benzoic acid (DABCYL); 4-dimethylaminophenylazophenyl-4′-isothiocyanate (DABITC); eosin; eosin isothiocyanate; erythrosin B; erythrosin isothiocyanate; ethidium; 5-carboxyfluorescein (FAM); 5-(4,6-dichlorotriazin-2-yl)aminofluorescein (DTAF); 2′,7′-dimethoxy-4′5′-dichloro-6-carboxyfluorescein (JOE); fluorescein; fluorescein isothiocyanate; fluorescamine; IR144; IR1446; Malachite Green isothiocyanate; 4-methylumbelliferone; ortho cresolphthalein; nitrotyrosine; pararosaniline; Phenol Red; phycoerythrin ((PE) including but not limited to B and R types); o-phthaldialdehyde; pyrene; pyrene butyrate; succinimidyl 1-pyrene butyrate; quantum dots; Reactive Red 4 (Cibacron™ Brilliant Red 3B-A); 6-carboxy-X-rhodamine (ROX); 6-carboxyrhodamine (R6G); lissamine rhodamine B sulfonyl chloride rhodamine; rhodamine B; rhodamine 123; rhodamine X isothiocyanate; sulforhodamine B; sulforhodamine 101; sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; and lanthanide chelate derivatives.

Particular fluorophores for use in the disclosed immunoassays include fluorescein, fluorescein isothiocyanate, phycoerythrin (PE), rhodamine B, and Texas Red (sulfonyl chloride derivative of sulforhodamine 101). Any of the fluorophores in the list preceding this paragraph can be used in the presently described assays, either to label the particle or bead, or to label a binding agent (e.g., an antibody or streptavidin). Fluorochromes can be attached by conventional covalent bonding, using appropriate functional groups on the fluorophores and on the particle or bead or binding agent (e.g., an antibody or streptavidin). The recognition of such groups and the reactions to form the linkages will be readily apparent to those skilled in the art. Other labels that can be used in place of the fluorophores are radioactive labels and enzyme labels. These are likewise known in the art. Flow cytometry methods and instrumentation are known in the art. Descriptions of instrumentation and methods can be found, e.g., in Introduction to Flow Cytometry: A Learning Guide (2000) Becton, Dickinson, and Company; McHugh, “Flow Microsphere Immunoassay for the Quantitative and Simultaneous Detection of Multiple Soluble Analytes,” Methods in Cell Biology 42, Part B (Academic Press, 1994).

The disclosed disclosure also pertains to kits and compositions for the detection of SARS-CoV-2 in a subject. The assay disclosed herein provides for the detection of SARS-CoV-2 or spike protein or fragments thereof in a biological sample selected from saliva and nasopharyngeal samples.

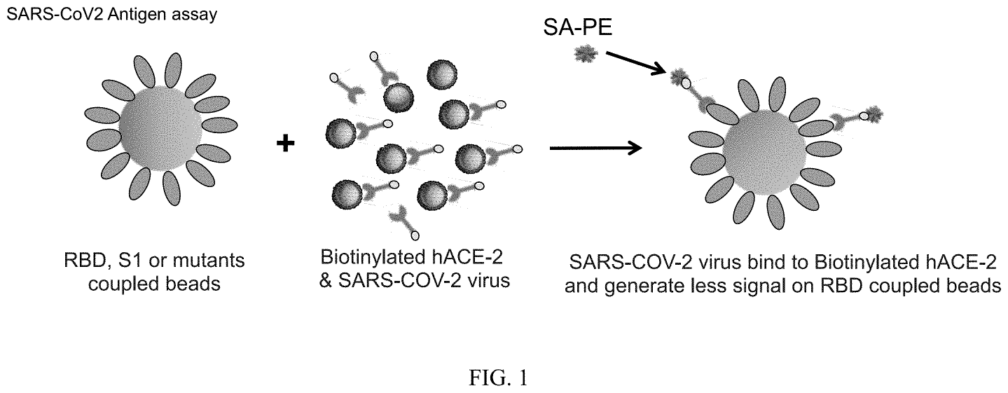

In one aspect of the disclosure, ACE2 receptor (preferably human ACE2 receptor and/or including truncated, modified ACE2 receptors, or ACE2 fusion proteins) can be used to identify the presence of SARS-CoV-2 and/or spike protein or fragments thereof in a biological sample selected from a saliva, throat, or a nasopharyngeal swab. This assay is illustrated in FIG. 1. In this aspect of the disclosure, the ACE2 receptor can be labeled or unlabeled. Thus, one embodiment provides a saliva, throat, or nasopharyngeal sample that is mixed with ACE2R. The ACE2R can be labeled with detectable label, biotinylated, or avidinated/streptavidinated. After a period of time, one or more subpopulations of beads onto which SARS-CoV-2 spike protein or fragments thereof are immobilized is added to the sample and ACE2R mixture. This population of beads is mixed (contacted) with mixture of ACE2 receptor and the sample, the population of beads is, optionally, washed and ACE2 receptor bound to the population of beads is then detected. If biotinylated ACE2R is used in the assay, streptavidin-PE is added to the population of beads to facilitate detection of ACE2R bound to the beads. The population of beads can be washed and then compared to control samples. A negative signal (the absence of a signal or a reduced signal for the binding of ACE2R to the beads) indicates the existence of SARS-CoV2 and/or spike protein or a fragment thereof capable of binding to ACE2R in the biological sample.

As discussed above, the assay can be conducted in the presence of one or more chaotropic agents. These agents can be used a concentration of about 0.1M to about 8M, when added to the coated solid supports to which antibodies are bound. Non-limiting examples of the chaotropic agents are sodium chloride, urea, guanidine hydrochloride, guanidine thiocyanate and/or or sodium thiocyanate. The effective concentrations of urea and/or sodium thiocyanate can be between 0.1M and 8M. The effective concentration of guanidine thiocyanate can be between 0.1M and 6M. The effective concentrations of sodium chloride can be between 0.1M and 5M. Detection of ACE2 receptor bound to beads can be performed using a Bio-Plex 2200, Bio-Plex 200 or Luminex platforms, such as LX-200, Magpix, Flexmap 360, etc. The identity of each assay is determined by the fluorescence signature of the dyed beads, and the amount of ACE2R captured by the beads comprising SARS-CoV-2 spike protein or a fragment thereof is determined by the fluorescence intensity of the attached labeled ACE2 receptor. The sample fluorescence intensity is compared to a set of standards or calibrators to generate a qualitative, semi-quantitative or quantitative result.

In the context of the disclosure, a fragment of any particular SARS-CoV-2 spike protein variant can comprise about 5 to about 50, about 10 to about 40, about 15 to about 30, about 20, about 10 or about 5 amino acids, provided that the span of amino acids includes one or more amino acid mutation identified in Table 1. As discussed above, fragments of a SARS-CoV-2 spike protein variant can range in length from 5 amino acids to (n−1) consecutive amino acids of the protein, where n is the total length of the SARS-CoV-2 spike protein, the S1 fragment of the spike protein or the S2 fragment of the spike protein. Thus, for the spike protein, the fragment length is between 5 and 1272 consecutive amino acids in length. In one embodiment, a fragment of the spike protein spans amino acids 13-1213 of the spike protein sequence. In another embodiment, the fragment is the RBD of the S1 protein or a fragment of the RBD. For the S1 protein (amino acids 13-685 of the disclosed spike protein length), a fragment is between 5 and 672 consecutive amino acids of the S1 sequence. For the S2 protein (amino acids 686-1273 of the disclosed spike protein), the fragment length is between 5 and 588 consecutive amino acids in length.

SARS-CoV-2 Spike Protein (1273 AA MW 141178)
SARS-CoV-2 S1 Protein (AA 13-685)
SARS-CoV-2 S2 Protein (AA 686-1273)
SEQ ID NO: 1
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVXNATRFASVYAWXRKRISNCVADYSXLYNSA
SFSTFKCYGVSPTKLNDLCFTNVYADSFVIRGDEVRQIAPGQTGKIADYNYKLPDDFTGCVI
XWNSNNLDSKVGGNYNYLYRLFRXSNLKPFERDISTEIYQAGSTPCNGXEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
XAXSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSA
IGKIQDSLSSTASALGKLQDVVNQNAQALNTLVKQLSSNFGAISSVLNDILSRLDXXEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLCCMTSC
CSCLKGCCSCGSCCKFDEDDSEPVLKGVKLHYT
>QJF75467.1 surface glycoprotein
[Severe acute respiratory syndrome coronavirus 2]
SARS-CoV-2 Spike Protein (1273 AA MW 141178)
SARS-CoV-2 S1 Protein (AA 13-685)
SARS-CoV-2 S2 Protein (AA 686-1273)
SEQ ID NO: 2
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVXNATRFASVYAWXRKRISNCVADYSXLYNSA
SESTFKCYGVSPTKLNDLCETNVYADSEVIRGDEVRQIAPGQTGKIADYNYKLPDDFTGCVI
XWNSNNLDSKVGGNYNYLYRLFRXSNLKPFERDISTEIYQAGSTPCNGXEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
XAXSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSA
IGKIQDSLSSTASALGKLQDVVNQNAQALNTLVKQLSSNFGAISSVLNDILSRLDXXEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLCCMTSC
CSCLKGCCSCGSCCKFDEDDSEPVLKGVKLHYT
YP_009724390.1 surface glycoprotein
[Severe acute respiratory syndrome coronavirus 2]
SARS-CoV-2 Spike Protein (1273 AA MW 141178)
SARS-CoV-2 S1 Protein (AA 13-685)
SARS-CoV-2 S2 Protein (AA 686-1273)
SEQ ID NO: 3
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
RAR/SVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFNFSQI
LPDPSKPSKRSFIEDLLFNKVTLADAGFIKQYGDCLGDIAARDLICAQKFNGLTVLPPLLTD
EMIAQYTSALLAGTITSGWTFGAGAALQIPFAMQMAYRFNGIGVTQNVLYENQKLIANQFNS
AIGKIQDSLSSTASALGKLQDVVNQNAQALNTLVKQLSSNFGAISSVLNDILSRLDKVEAEV
QIDRLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTORNFYEPQII
TTDNTFVSGNCDVVIGIVNNTVYDPLQPELDSFKEELDKYFKNHTSPDVDLGDISGINASVV
NIQKEIDRLNEVAKNLNESLIDLQELGKYEQYIKWPWYIWLGFIAGLIAIVMVTIMLCCMTS
CCSCLKGCCSCGSCCKFDEDDSEPVLKGVKLHYT
ACE2 [Homo sapiens]
GenBank: BAB40370.1
>BAB40370.1 ACE2 [Homo sapiens]
SEQ ID NO: 4
MSSSSWLLLSLVAVTAAQSTIEEQAKTFLDKFNHEAEDLFYQSSLASWNYNTNITEENVQNM
NNAGDKWSAFLKEQSTLAQMYPLQEIQNLTVKLQLQALQQNGSSVLSEDKSKRLNTILNTMS
TIYSTGKVCNPDNPQECLLLEPGLNEIMANSLDYNERLWAWESWRSEVGKQLRPLYEEYVVL
KNEMARANHYEDYGDYWRGDYEVNGVDGYDYSRGQLIEDVEHTFEEIKPLYEHLHAYVRAKL
MNAYPSYISPIGCLPAHLLGDMWGRFWTNLYSLTVPFGQKPNIDVTDAMVDQAWDAQRIFKE
AEKFFVSVGLPNMTQGFWENSMLTDPGNVQKAVCHPTAWDLGKGDFRILMCTKVTMDDFLTA
HHEMGHIQYDMAYAAQPFLLRNGANEGFHEAVGEIMSLSAATPKHLKSIGLLSPDFQEDNET
EINFLLKQALTIVGTLPETYMLEKWRWMVFKGEIPKDQWMKKWWEMKREIVGVVEPVPHDET
YCDPASLFHVSNDYSFIRYYTRTLYQFQFQEALCQAAKHEGPLHKCDISNSTEAGQKLFNML
RLGKSEPWTLALENVVGAKNMNVRPLLNYFEPLFTWLKDQNKNSFVGWSTDWSPYADQSIKV
RISLKSALGDRAYEWNDNEMYLFRSSVAYAMRQYFLKVKNQMILFGEEDVRVANLKPRISFN
FFVTAPKNVSDIIPRTEVEKAIRMSRSRINDAFRLNDNSLEFLGIQPTLGPPNQPPVSIWLI
VFGVVMGVIVVGIVILIFTGIRDRKKKNKARSGENPYASIDISKGENNPGFQNTDDVQTSF
Exemplary ACE2-Fc fusion protein
SEQ ID NO: 5
QSTIEEQAKTFLDKFNHEAEDLFYQSSLASWNYNTNITEENVQNMNNAGDKWSAFLKEQSTL
AQMYPLQEIQNLTVKLQLQALQQNGSSVLSEDKSKRLNTILNTMSTIYSTGKVCNPDNPQEC
LLLEPGLNEIMANSLDYNERLWAWESWRSEVGKQLRPLYEEYVVLKNEMARANHYEDYGDYW
RGDYEVNGVDGYDYSRGQLIEDVEHTFEEIKPLYEHLHAYVRAKLMNAYPSYISPIGCLPAH
LLGDMWGRFWTNLYSLTVPFGQKPNIDVTDAMVDQAWDAQRIFKEAEKFFVSVGLPNMTQGF
WENSMLTDPGNVQKAVCHPTAWDLGKGDFRILMCTKVTMDDFLTAHHEMGHIQYDMAYAAQP
FLLRNGANEGFHEAVGEIMSLSAATPKHLKSIGLLSPDFQEDNETEINFLLKQALTIVGTLP
FTYMLEKWRWMVFKGEIPKDQWMKKWWEMKREIVGVVEPVPHDETYCDPASLFHVSNDYSFI
RYYTRTLYQFQFQEALCQAAKHEGPLHKCDISNSTEAGQKLFNMLRLGKSEPWTLALENVVG
AKNMNVRPLLNYFEPLFTWLKDQNKNSFVGWSTDWSPYADQSIKVRISLKSALGDKAYEWND
NEMYLFRSSVAYAMRQYFLKVKNQMILFGEEDVRVANLKPRISFNFFVTAPKNVSDIIPRTE

VEKAIRMSRSRINDAFRLNDNSLEFLGIQPTLGPPNQPPVSIGGGGGGGGGTHTCPPCPAPE
LLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQ
GNVFSCSVMHEALHNHYTQKSLSLSPGK

### EXAMPLES

### Example 1—Measurement of Neutralizing Antibodies Specific for SARS-CoV-2 Spike Protein Variants or Fragments Thereof

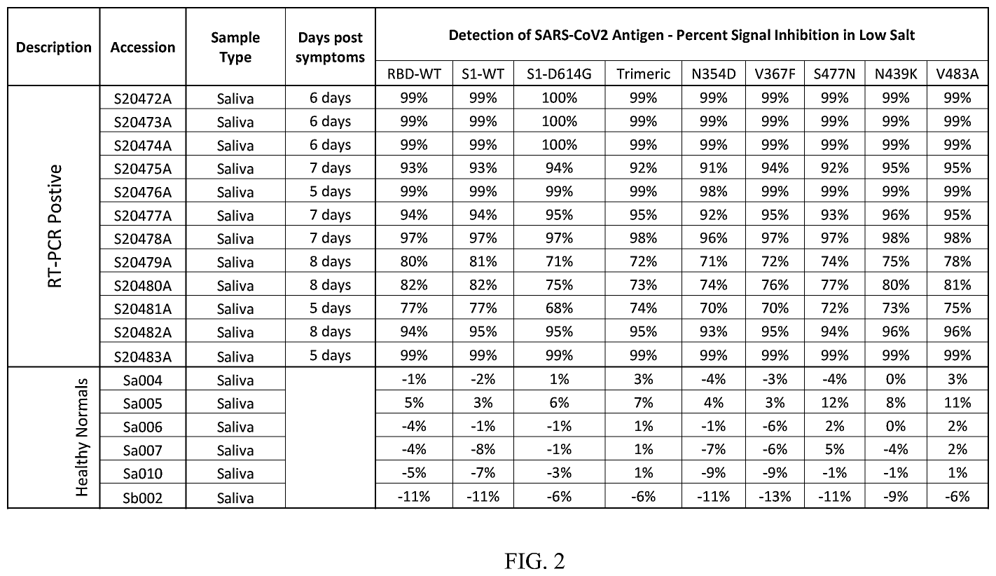

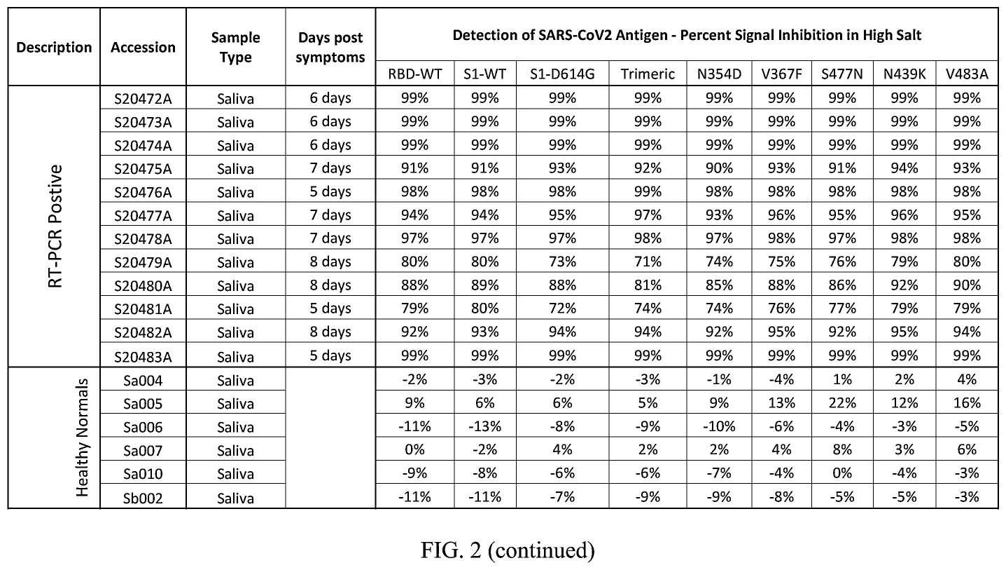

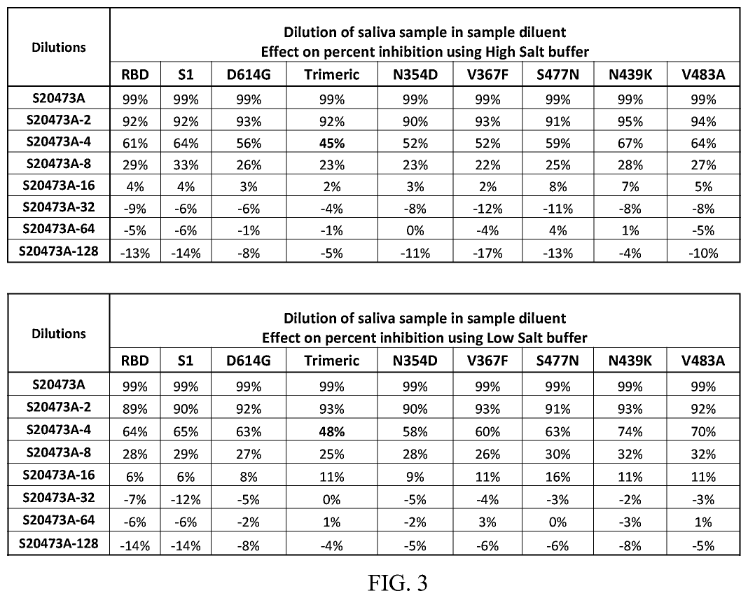

A saliva sample from an individual is mixed with biotinylated human ACE2R and incubated together for a period of time. A population of fluorescently labeled beads comprising a plurality of subpopulations of fluorescently labeled beads to which individual SARS-CoV-2 spike protein variants or fragments thereof were immobilized was then added to the saliva sample. Unbound biotinylated human ACE2 receptor was washed away and labeled streptavidin-PE (Phycoerythrin) was added. The reaction was incubated and then washed prior to detection using a Bio-Plex 2200, Bio-Plex 200 or Luminex LX-200 platform. The sample fluorescence intensity was compared to the fluorescence intensity of a set of standards or calibrators to generate a qualitative result. A lack of signal or reduced signal for the population of beads onto which a SARS-CoV-2 spike protein variant or fragment thereof was immobilized indicates that SARS-CoV-2 is present in the saliva sample. FIGS. 2 and 3 illustrate this concept. In FIG. 2, SARS-CoV-2 present in the saliva samples is shown by the high levels of signal inhibition in samples obtained from SARS-CoV-2 samples obtained from patients having a positive RT-PCR result. Similar results were observed under both high salt and low salt conditions. Healthy individually show no inhibition of signal under high salt or low salt conditions (indicating a lack of SARS-CoV-2 in the sample).

In FIG. 3, the effects of sample dilution were tested. As is apparent from the data, inhibition of signal could be diluted out of the tested sample under both high salt and low salt conditions which indicates the presence of little, or no, SARS-CoV-2 in the diluted sample. For example, a 1:32 dilution of the sample exhibited no signal inhibition and a 1:16 dilution of the sample exhibited minimal signal inhibition. Undiluted (neat) samples, and samples diluted 1:2, 1:4 and 1:8 exhibited substantially similar signal inhibition across all tested spike protein variants thus indicating the applicability of the assay across various SARS-CoV-2 variants.

We claim:

1. A method for detecting SARS-CoV-2 in a mammal comprising:
a) obtaining a biological sample comprising saliva, a throat swab, a nasopharyngeal sample, or a nasal mucosal sample from the mammal;
b) admixing the biological sample with labeled angiotensin-converting enzyme 2 receptor (ACE2R) to form a first admixture;
c) admixing a solid substrate to which spike protein or fragments thereof are immobilized to form a second admixture;
d) separating the solid substrate from components of the second admixture;
e) optionally washing the separated solid substrate; and
f) detecting ACE2R bound to the spike protein or fragments thereof immobilized on the solid substrate.

2. The method according to claim 1, wherein the mammal is a human, non-human primate.

3. The method according to claim 1, wherein ACE2 receptor binding to a substrate or substrate subpopulation is detected using an antibody specific for ACE2 receptor that is detectably labeled.

4. The method according to claim 1, wherein ACE2 receptor binding to a substrate subpopulation is detected using ACE2 receptor that is detectably labeled.

5. The method according to claim 3, wherein the ACE2 receptor is labeled with phycoerythrin (PE).

6. The method according to claim 1, wherein the ACE2 receptor is biotinylated and detected using streptavidin-PE.

7. The method according to claim 1, wherein fluorescence intensity of ACE2R bound to the substrate is compared to a set of standards or calibrators to generate a qualitative, semi-quantitative or quantitative result.

8. The method according to claim 1, wherein the method is performed in the presence of one or more chaotropic agent.

9. The method according to claim 8, wherein the chaotropic agent is sodium chloride, sodium thiocyanate, and/or urea.

10. The method according to claim 9, wherein the chaotropic agent is sodium chloride.

11. The method according to claim 10, wherein the concentration of sodium chloride is between: 0.5 M to about 2.0 M, about 0.75 M to about 1.25 M, or about 1.0 M.

12. The method according to claim 10, wherein the concentration of sodium chloride is between 0.0 M to 0.5 M, about 0.1 M and about 0.2 M, or about 0.15 M.

13. The method according to claim 1, wherein ACE2 receptor binding to the substrate is detected using ACE2 receptor that is detectably labeled or with an antibody specific for the ACE2 receptor that is detectably labeled.

14. The method according to claim 1, wherein the ACE2 receptor is labeled with phycoerythrin (PE) or is biotinylated and detected using streptavidin-PE.

15. The method according to claim 14, wherein the ACE2 receptor is biotinylated and detected using streptavidin-PE.

16. The method according to claim 1, wherein the biological sample is saliva.

17. The method according to claim 1, wherein the SARS-CoV-2 spike protein or fragment thereof that is immobilized on the substrate comprises one or more of the following:
a) a SARS-CoV-2 spike protein variant or fragment thereof containing the mutation V367X, where X is any amino acid or X is F;
b) a SARS-CoV-2 spike protein variant or fragment thereof containing the mutation F342X, where X is any amino acid or X is L;
c) a SARS-CoV-2 spike protein variant or fragment thereof containing the mutation A435X, where X is any amino acid or X is S;
d) a SARS-CoV-2 spike protein variant or fragment thereof containing the mutation K458X, where X is any amino acid or X is R;
e) a SARS-CoV-2 spike protein variant or fragment thereof containing the mutation V483X, where X is any amino acid or X is A;
f) a SARS-CoV-2 spike protein variant or fragment thereof containing the mutation V483X, where X is any amino acid or X is A;
g) a SARS-CoV-2 spike protein variant or fragment thereof containing the mutation N354X, where X is any amino acid or X is D;
h) a SARS-CoV-2 spike protein variant or fragment thereof containing the mutations R683X1 and/or R685X2, where X1 and X2 are any amino acid or X1 and X2 are A;
i) a SARS-CoV-2 spike protein variant or fragment thereof containing the mutations K986X3 and/or V987X4, where X3 and X4 are any amino acid or X3 and X4 are P;
j) a SARS-CoV-2 spike protein variant or fragment thereof containing the mutations R683X1, R685X2, K986X3, and V987X4, where X1 is any amino acid or X1 is A; X2 is any amino acid or X2 is A; X3 is any amino acid or X3 is P; and X4 is any amino acid or X4 is P;
k) a wild-type SARS-CoV-2 spike protein or fragment thereof;
l) a SARS-CoV-2 spike protein or fragment thereof derived from SARS-CoV-2 variant B.1.1.7;
m) a SARS-CoV-2 spike protein or fragment thereof derived from SARS-CoV-2 variant B.1.351; or
n) a SARS-CoV-2 spike protein or fragment thereof derived from SARS-CoV-2 variant P.1.

18. The method according to claim 1, wherein said substrate comprises a population of particles or beads, said population comprises one or more separate subpopulations of particles or beads selected from:
a) a SARS-CoV-2 spike protein variant or fragment thereof containing the mutation V367X immobilized on a first particle or bead having a first specific detectable physical parameter, where X is any amino acid or X is F;
b) a SARS-CoV-2 spike protein variant or fragment thereof containing the mutation F342X immobilized on a second particle or bead having a second specific detectable physical parameter, where X is any amino acid or X is L;
c) a SARS-CoV-2 spike protein variant or fragment thereof containing the mutation A435X immobilized on a third particle or bead having a third specific detectable physical parameter, where X is any amino acid or X is S;
d) a SARS-CoV-2 spike protein variant or fragment thereof containing the mutation K458X immobilized on a fourth particle or bead having a fourth specific detectable physical parameter, where X is any amino acid or X is R;
e) a SARS-CoV-2 spike protein variant or fragment thereof containing the mutation V483X immobilized on a fifth particle or bead having a fifth specific detectable physical parameter, where X is any amino acid or X is A;
f) a SARS-CoV-2 spike protein variant or fragment thereof containing the mutation V483X immobilized on a sixth particle or bead having a sixth specific detectable physical parameter, where X is any amino acid or X is A;
g) a SARS-CoV-2 spike protein variant or fragment thereof containing the mutation N354X immobilized on a seventh particle or bead having a seventh specific detectable physical parameter, where X is any amino acid or X is D;
h) a SARS-CoV-2 spike protein variant or fragment thereof containing the mutations R683X1 and/or R685X2 immobilized on an eighth particle or bead having an eighth specific detectable physical parameter, where X1 and X2 are any amino acid or X1 and X2 are A;
i) a SARS-CoV-2 spike protein variant or fragment thereof containing the mutations K986X3 and/or V987X4 immobilized on a ninth particle or bead having a ninth specific detectable physical parameter, where X3 and X4 are any amino acid or X3 and X4 are P;
j) a SARS-CoV-2 spike protein variant or fragment thereof containing the mutations R683X1, R685X2, K986X3, and V987X4 immobilized on a tenth particle or bead having a tenth specific detectable physical parameter, where X1 is any amino acid or X1 is A; X2 is any amino acid or X2 is A; X3 is any amino acid or X3 is P; and X4 is any amino acid or X4 is P;
k) a wild-type SARS-CoV-2 spike protein or fragment thereof immobilized on an eleventh particle or bead having an eleventh specific detectable physical parameter;
l) a SARS-CoV-2 spike protein or fragment thereof derived from SARS-CoV-2 variant B.1.1.7 immobilized on a twelfth particle or bead having a twelfth specific detectable physical parameter;
m) a SARS-CoV-2 spike protein or fragment thereof derived from SARS-CoV-2 variant B.1.351 immobilized on a thirteenth particle or bead having a thirteenth specific detectable physical parameter; and
n) a SARS-CoV-2 spike protein or fragment thereof derived from SARS-CoV-2 variant P.1 immobilized on a fourteenth particle or bead having a fourteenth specific detectable physical parameter.

19. The substrate according to claim 18, wherein said population of particles or beads comprises two, three, four, five, six, seven, eight, nine, ten or more subpopulations of particles or beads, each subpopulation having a specific detectable physical parameter.

20. The substrate according to claim 18, wherein the specific detectable physical parameter is a fluorescent dye, luminescent agent, electron-dense reagent, radioisotope or particle size.

21. The substrate according to claim 20, wherein the specific detectable parameter is a fluorophore.
